(19) United States
(12) Patent Application Publication
(10) Pub. No.: US 2022/0250057 A1
(43) Pub. Date: Aug. 11, 2022

(54) MICROFLUIDIC CHIP FOR STRUCTURING CELL AGGREGATES BY OPTICAL EXCLUSION AND ACOUSTIC LEVITATION

(71) Applicants: CENTRE NATIONAL DE LA RECHERCHE SCIENTIFIQUE, Paris (FR); ECOLE SUPERIEURE DE PHYSIQUE ET DE CHIMIE INDUSTRIELLES DE LA VILLE DE PARIS, Paris 05 (FR); SORBONNE UNIVERSITE, Paris (FR); UNIVERSITÉ DE PARIS, Paris (FR)

(72) Inventors: Jean-luc AIDER, Vanves (FR); Mauricio HOYOS, Creteil (FR); Nathan JEGER-MADIOT, Paris (FR)

(21) Appl. No.: 17/595,723

(22) PCT Filed: Jun. 5, 2020
(86) PCT No.: PCT/EP2020/065732

§ 371 Date: Nov. 23, 2021

(30) Foreign Application Priority Data
Jun. 6, 2019 (FR) 1906031

(51) Int. Cl. B01L 3/00

(52) U.S. Cl. CPC B01L 3/50273; B01L 3/502761; B01L 2400/0454; B01L 2300/163; B01L 2400/0436; B01L 2200/0652

(57) ABSTRACT

A microfluidic chip, in particular for a cell culture, the chip including a block made from biocompatible material, a passage channel made in the block for the passage of cells bathed in a liquid, in particular a nutrient liquid, a resonant cavity made in the block, connected to the passage channel and including walls for containing the cells originating from the passage channel, a generator generating acoustic waves capable of forming at least one cell aggregate in acoustic levitation in the resonant cavity, and at least one optical emitter capable of illuminating cells in the resonant cavity through at least one wall of the resonant cavity and simultaneous to the generation of acoustic waves in such a way as to structure the at least one aggregate by means of the optical exclusion technique.

The present invention relates to a microfluidic chip for manipulating objects such as micro- or nanoparticles with a size comprised between 0.1 μm and 300 μm. More particularly, it relates to the levitation of cells by applying acoustic waves. The following refers mainly to cells, but the present invention applies to any particle sensitive at least to acoustic waves.

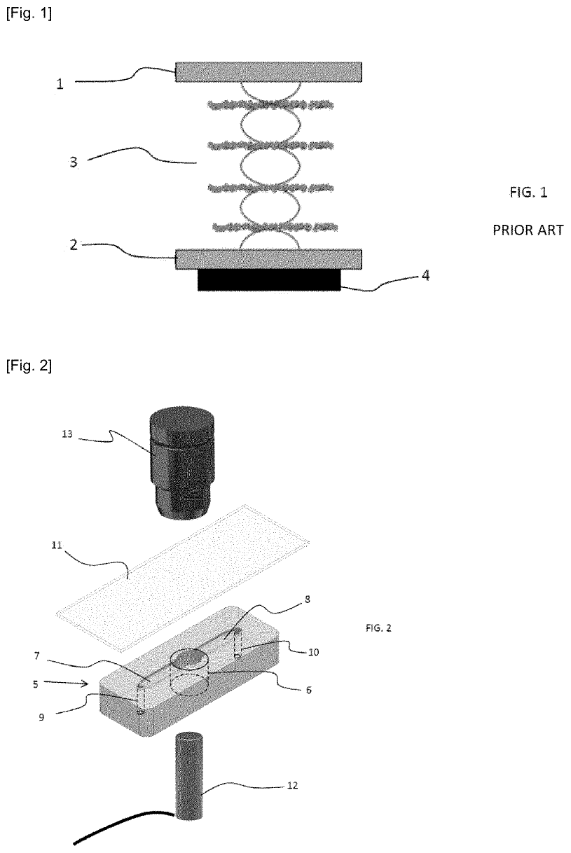

In a general way, the principle of acoustic levitation and the formation of particle (or cell) aggregates in acoustic levitation is recapped in FIG. 1. Depending on the frequency, it is possible to generate 1 to N pressure nodes between the two opposite walls 1 and 2 of a channel 3. The condition to be met is h=N·λ_{ac}/2, where h is the height of the cavity and λ_{ac} is the ultrasonic wavelength (acoustic wave). A transducer 4 is arranged outside the channel in contact with the wall 2 so as to generate acoustic waves through this wall 2. The wall 1 is a reflector which reflects the acoustic waves in the channel 3 so as to generate a standing wave and N pressure nodes distributed over the height of the cavity. These variations in pressure result in the creation of an acoustic radiation force field. The axial component of the acoustic radiation force created in this way will then promote the formation of aggregates in each of the pressure nodes which define acoustic levitation planes. The cells are distributed over several aggregates, each aggregate is arranged on a pressure node defining a plane. The lines made in FIG. 1 merely indicate the acoustic radiation force resulting in the pressure nodes.

More precisely, under the effect of the acoustic radiation force, particles (or cells) in suspension are forced to migrate towards the acoustic pressure nodes. Once in the different acoustic levitation planes, the radial component of the acoustic radiation force forces the particles to collect together and form an aggregate at each level. The frequency of the ultrasonic wave is defined so as to obtain several pressure nodes distributed over the height h of the channel.

In current acoustofluidic applications, the acoustic radiation force is used to separate the species as a function of their physical and mechanical properties, namely their density and their compressibility, as well as their size, since the acoustic force is directly dependent on these parameters, as defined in the following equation:

F_{ac}=4π·r³·k_{ac}·E₀·Φ·sin(2k_{ac}·z)

Where r is the radius of the particle, Φ is the acoustic contrast factor, k_{ac} is the acoustic wavenumber and E₀ is the acoustic energy. This defines the sensitivity of the particles (or cells) to the acoustic radiation force. The denser and larger the particle is, the greater the acoustic force that it is subjected to is and the faster the particle migrates towards the pressure node. This force is usually used to separate flowing particles or cells as a function of these characteristics.

Document WO 2019/002551 A1 is known, which describes a method for separating flowing particles by applying acoustic waves in order to levitate them. This document also describes the principle of optical exclusion. The present invention refers to this document WO 2019/002551 A1 for the methods of levitating the particles and for the application of the principle of optical exclusion.

The object of the present invention is a novel use of the principle of acoustic levitation with optical manipulation.

Another object of the invention is a device making an optimized manipulation of aggregates created by acoustic levitation possible.

Yet another object of the invention is a novel method for creating the aggregates.

At least one of the above-named objectives is achieved with a microfluidic chip comprising:
a block made from biocompatible material,
a passage channel made in the block for the passage of cells bathed in a liquid, in particular a nutrient liquid (culture medium),
a resonant cavity, for example intended for cell culture, made in the block, connected to the passage channel and comprising walls for containing the cells originating from the passage channel,
an acoustic wave generator capable of forming at least one cell aggregate in acoustic levitation in the resonant cavity,
at least one optical emitter capable of illuminating cells in the resonant cavity through at least one wall of the resonant cavity and simultaneous with the generation of acoustic waves in such a way as to structure said at least one aggregate by means of the technique of optical exclusion.

The resonant cavity comprises walls for containing the cells. In other words, the cells can penetrate an area in the resonant cavity where they are no longer affected by the flow in the passage channel.

They can therefore be subjected to the action of the acoustic and optical waves.

They can also remain immobile, relative to the movement of the flow in the passage channel. Immobile means that they are subjected to small movements of the medium, but not to the linear displacement movement of the flow.

The rate of the flow can be adjusted so that it does not involve the cells forming in particular one or more aggregates. The acoustic force is then great enough to oppose the flow.

With the microfluidic chip according to the invention, it is possible to use the acoustic waves and the optical beams simultaneously so as to hold the cells in levitation over one or more layers by acoustic waves while manipulating them by optical beams according to the technique of optical exclusion.

The microfluidic chip according to the invention constitutes an optoacoustic bioreactor in which the cells can be cultured in acoustic levitation and manipulated by means of specific illumination so as to structure the aggregates in successive layers of cells. By structuring is meant a spatial distribution of the layers of cells which are different from one another.

In the resonant cavity, the cells are bathed in a liquid which does not disrupt the acoustic waves or the optical beams used and is suitable for cell culture.

The resonant cavity is advantageously intended for a culture of cells in acoustic levitation. It is clear to a person skilled in the art that any micro- or nanoparticle that is sensitive to acoustic waves and possibly to certain optical beams, endogenously or exogenously, can be used in the invention.

Using both optical and acoustic properties, therefore, it is possible to spatially structure cell aggregates in acoustic levitation. This operation can be repeated several times, by successive injections of different types of cells, which are marked (fluorescent biological markers) or not, and which react to different optical wavelengths.

The acoustic radiation force due to the acoustic waves makes it possible to create particle aggregates which can be held in acoustic levitation for as long as necessary. This force is dimensioned in order to hold this aggregate in acoustic levitation in the resonant cavity, which constitutes an acoustic trap.

According to an advantageous characteristic of the invention, the microfluidic chip can moreover comprise at least one second optical emitter, the two emitters being arranged so as to emit through two opposite walls of the resonant cavity respectively. Preferably, the two optical emitters emit at different wavelengths. Such an arrangement makes it possible to act, by means of the technique of optical exclusion, on different types of cells simultaneously or sequentially. This also makes it possible to act on aggregates symmetrically because there is an attenuation of the intensity of the beams passing through a thickness of fluid and encountering aggregates along its axis of propagation. Owing to this setup, the top aggregate is acted on in the same way as the bottom aggregate.

According to an embodiment of the invention, the acoustic wave generator can comprise an upper element and a lower element sandwiching at least part of the resonant cavity on two opposite walls; the upper element, which is fixed or removable, being an upper transducer or an acoustic wave reflector; and the lower element being a lower transducer, the upper and lower transducers being capable of emitting acoustic waves. Furthermore, the upper element and/or the lower element are transparent to the light beams provided for illuminating the cells.

In other words, the generator comprises a lower transducer combined with a reflector capable of reflecting the acoustic waves from the lower transducer so as to create the acoustic radiation force in the resonant cavity. However, the reflector can be replaced with an upper transducer generating the same acoustic waves as the lower transducer in a synchronous manner so as to also create the acoustic radiation force in the resonant cavity.

Preferably, at least one of the two transducers is transparent to the optical wavelength or else is annular in order to allow the optical illumination to pass into its centre.

The removable nature of the upper element may make it possible to evacuate or recover the cells forming aggregates.

According to an embodiment of the invention, the resonant cavity is closed at its lower end by the lower transducer. The lower transducer thus constitutes the lower wall of the resonant cavity. In such an embodiment, the lower transducer is in direct contact with the culture medium. This configuration makes it possible to improve the energy efficiency of the resonant cavity.

According to an advantageous characteristic of the invention, the lower transducer can be arranged inside the resonant cavity.

A removable lower transducer capable of sliding inside the resonant cavity is thus provided. Preferably, the dimensions of the transducer make it possible to ensure that the resonant cavity is sealed. In this case, the usable volume forming the culture medium is smaller than the total volume of the resonant cavity. It is thus possible to define different volumes of the resonant cavity. It is thus a cavity with a variable volume and therefore a variable number of levitation planes.

Advantageously, the resonant cavity can have at least one stop for blocking the head of the lower transducer once it has been inserted in the resonant cavity.

According to an embodiment of the invention, the resonant cavity can be closed at its lower end by a fixed or removable film which is transparent to the acoustic waves originating from the lower transducer arranged outside the resonant cavity.

This film can be made from polydimethylsiloxane (PDMS) material or of cyclic olefin copolymer (COC) or can be an adhesive PCR (Polymerase Chain Reaction) film which is easily detachable and makes a very good transfer of the ultrasonic waves possible. A removable film makes the passage or recovery of cell aggregates at the end of culturing possible, for example.

Preferably, the resonant cavity is a cylinder the side walls of which are constituted by the block. The upper base can be formed by the reflector or the upper transducer. The lower base can be formed by a film or directly by the lower transducer.

According to an embodiment, the passage channel can lead into the resonant cavity at the upper end of a side wall of the cylinder. Such an arrangement makes it possible to supply cells to the cavity through the upper end of the side wall of the cylinder.

According to the invention, the resonant cavity can have a stop arranged so that the head of the lower transducer can be inserted to reduce the height of the usable volume in the resonant cavity until it is equal to the height of the passage channel.

Ideally, the usable volume makes it possible to produce one or more monolayer or multi-layer cell aggregates.

According to an advantageous embodiment of the invention, the passage channel can be made on the upper surface of the block and a bonded or removable strip can cover all of the surface of the block, including the upper end of the resonant cavity; this strip being transparent to the optical beams provided to illuminate the cells of the resonant cavity from the outside. In addition, when a transducer is arranged opposite it, this strip reflects the acoustic waves from the transducer on the internal side of the resonant cavity.

Preferably, the passage channel and the cylinder can be arranged perpendicular to one another. The block can then moreover comprise two microchannels passing through the block from one side to the other, parallel to the cylinder and connected respectively to the two free ends of the passage channel; the first microchannel being intended for the arrival of cells in the passage channel and the second microchannel being intended for evacuating cells from the passage channel.

By way of non-limitative example, the reflector is a strip made from glass, from polymethyl methacrylate (PMMA), from quartz, from silicon, from polydimethylsiloxane (PDMS) or from cyclic polyolefin copolymer (COC). Such a strip is designed to ensure a good transmission on the one hand and a good reflection on the other. Preferably, the reflector can be designed starting from a material identical to that of the block and having an internal surface treated to reflect acoustic waves.

The chip according to the invention can comprise several microchannels made in the thickness of the block and leading into the resonant cavity for cells to enter and/or exit. Preferably, these microchannels are aligned respectively with the pressure nodes provided in the resonant cavity because of the acoustic radiation force. These microchannels can be used, for example, to inject cells of different natures from those injected through the passage channel, to inject nutrients, and also to evacuate cells.

Advantageously, the height of the resonant cavity can be a function of the number of pressure nodes to be created and the wavelength of the acoustic waves generated by the generator. The resonant cavity is designed to contain a superimposition of several cell layers, while the height of the passage channel is preferably adapted to the size of a cell.

The resonant cavity is taller than the passage channel, for example several times the diameter of a cell, in particular at least 10 times the diameter. It is possible to try to put the first pressure node at the level of the passage channel, therefore a channel height of the order of λ/4.

Preferably, the passage channel can have a rectilinear shape, such that the section of the passage channel between an inlet end and the resonant cavity is colinear with the section of the passage channel between the resonant cavity and the other, outlet, end of the passage channel.

According to an embodiment of the invention, the block can be made from polydimethylsiloxane (PDMS) or from cyclic olefin copolymer (COC).

In particular, the block is designed starting from a gas-permeable material in order to facilitate the gas exchanges between the medium, the contents of the resonant cavity, and the outside if necessary. The channels are etched or moulded into the block and can be installed in an incubator so as to ensure the optimum culture conditions in terms of gas and temperature.

In particular, the resonant cavity can be dimensioned with a height greater than the diameter of the passage channel leading into this resonant cavity. Thus, the passage channel, which is a conduit with a small diameter, typically suitable for conveying the cells only in a line, is clearly distinguished from the resonant cavity, which has a larger dimension capable of containing cell aggregates in one plane and in one volume.

By way of non-limitative example, the resonant cavity can have a diameter between 1 and 50 mm, a height of the resonant cavity comprised between 5 and 15 mm and a height of the passage channel equal to 450 μm.

The microfluidic chip according to the invention can moreover comprise at least one additional microchannel made in the block in the same plane as the passage channel.

This is a simplified arrangement which makes it possible to supply cells to and evacuate cells from the resonant cavity, which cells can form aggregates in the same plane as the cells originating from the passage channel, which is, in fact, a main microchannel.

This additional microchannel can also be used to inject other cells, biomarkers, or else to wash the culture medium or recover the production of the cells during their culturing.

According to another aspect of the invention, a method for manipulating cells in acoustic levitation in a microfluidic chip as defined above is proposed. This method comprises the following steps:
injecting cells into the resonant cavity via an inlet of the passage channel,
generating acoustic waves for acoustically levitating the injected cells so as to form a cell aggregate in at least one layer,
at least one phase of illuminating, at a specific optical wavelength, the cells while simultaneously maintaining the acoustic waves so as to manipulate cells according to the optical exclusion principle.

According to the invention, the selective opto-acousto-fluidic exclusion principle is used, which describes the fact that one particle or cell reacts to certain optical wavelengths while other particles or cells do not react at all.

According to an advantageous embodiment of the invention, the injected cells have different natures, and the steps of generating acoustic waves and illuminating can be carried out as follows:
generating acoustic waves for acoustically levitating the injected cells and at the same time applying a light beam at a specific wavelength making the principle of exclusion of the cells sensitive to this wavelength possible, so that only the cells not sensitive to this optical wavelength form an aggregate in one layer,
maintaining the acoustic waves and stopping the light beam so that the cells sensitive to the wavelength of the light beam now form aggregates on the periphery of the aggregate already formed, to thus obtain a radially structured aggregate.

The present invention thus makes it possible to produce radially structured monolayer cell aggregates. The aggregate can thus be composed of successive rings of different cells, and thus make the co-culture of cells possible, steps which are indispensable for the formation of organoids.

By cell aggregate is meant a layer of cells satisfying all of the following characteristics: at least two cells included in said layer, in particular at least 10%, better 25%, preferably 50% of the cells included in said layer, are in contact, and said layer has, over at least part of its length, a succession of cells during the displacement in at least one of its transverse dimensions.

According to an advantageous embodiment of the invention, a three-dimensional structure formed of several layers of aggregates is produced by carrying out the following steps:
the step of injecting comprises the injection of cells into the resonant cavity via one or more inlets, and
the step of generating acoustic waves moreover comprises the generation of acoustic waves for acoustically levitating several aggregates of cells injected on several levels, the levels being acoustic pressure nodes the number of which depends on the wavelength of the acoustic waves and the height of the resonant cavity.

The invention makes it possible to create several monolayers (cell sheets) one above another, which brings a considerable time saving for the cell culture since it is no longer necessary to trypsinize a sheet of cells, to detach it from a possible solid substrate in order then to deposit it on another. This process is more particularly suitable for MSC-type (Mesenchymal Stromal Cells) stem cell cultures, where the cells are cultured to manufacture cell sheets which are then superimposed, but it is relevant to any type of cell culture intended to form cell sheets.

With the invention, it is possible to envisage the manufacture, in acoustic levitation, of spheroids, organoids or tumoroids which could be held and cultured in acoustic levitation.

The invention makes it possible to carry out a cell culturing while holding the aggregate or the aggregates obtained immobile in acoustic levitation for the duration of the culturing.

Such a system has thus made it possible to culture MSCs in acoustic levitation for 18 days. At the end of this culturing, the cells were living and could proliferate normally once deposited on a substrate. The system has thus been validated for culturing over short periods (a few hours) and over long periods (a few weeks). This is made possible owing to the flow of nutrients and the porosity of the materials used, which makes the gas exchanges in an incubator possible. The acoustic bioreactor was placed in an incubator in order to make the culturing of cells possible. The compactness of the present system makes it possible to place several chips in an incubator. It is thus possible to carry out the culturing of a number of sheets or spheroids in acoustic levitation in parallel (a few tens per well, and a few wells in the incubator).

Other characteristics and advantages of the invention will become apparent on reading the detailed description of implementations and embodiments which are in no way limitative, in the light of the attached figures, in which:

FIG. 1 is a diagrammatic view of the creation of pressure nodes in a channel by means of acoustic levitation according to the prior art;

FIG. 2 is a diagrammatic exploded view of an example of the device according to the invention,

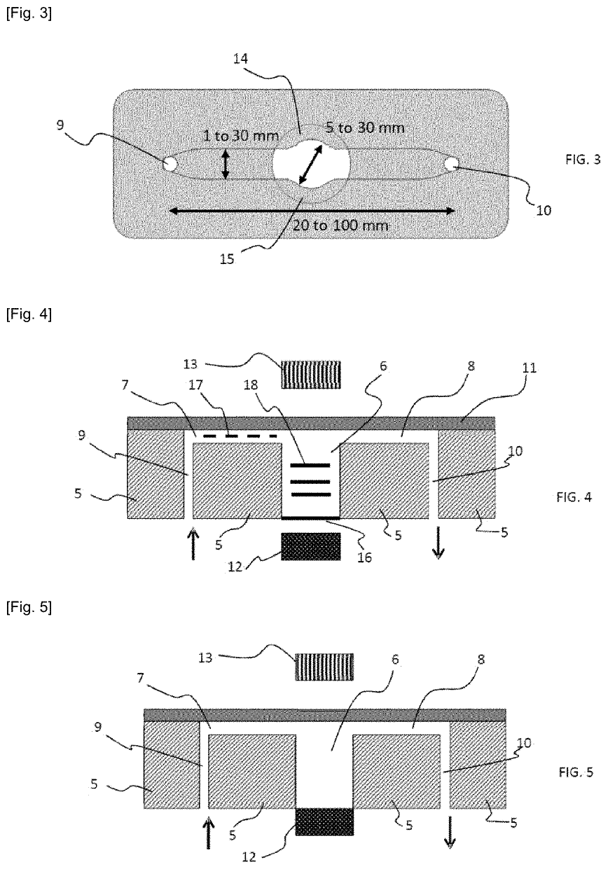

FIG. 3 is a view from below of an example of the device according to the invention,

FIG. 4 is a diagrammatic sectional view along the longitudinal axis of the passage channel, with an optical emitter and an acoustic wave transducer arranged outside the resonant cavity, according to the invention,

FIG. 5 is a diagrammatic sectional view along the longitudinal axis of the passage channel, with an optical emitter and an acoustic wave transducer closing the resonant cavity on its base, according to the invention,

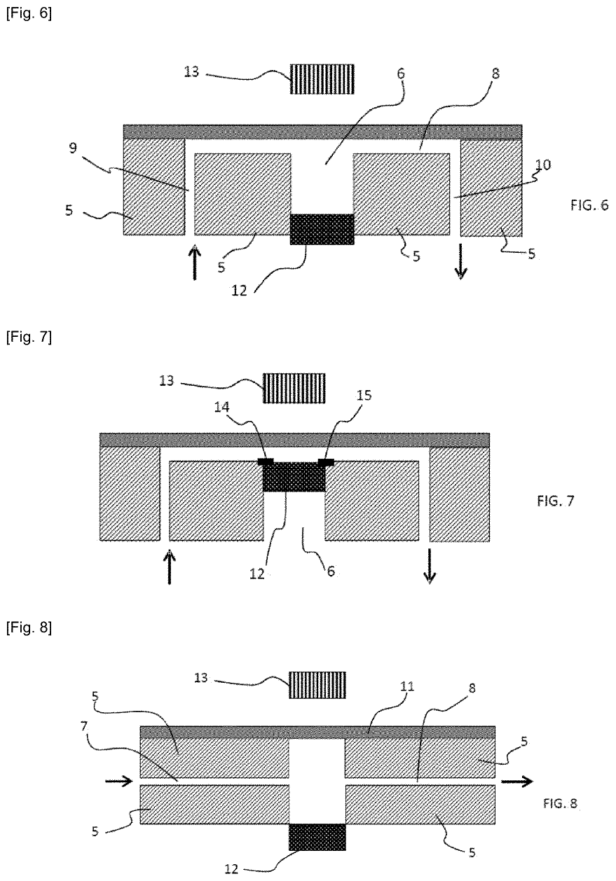

FIG. 6 is a diagrammatic sectional view along the longitudinal axis of the passage channel, with an optical emitter and an acoustic wave transducer arranged inside the resonant cavity, according to the invention,

FIG. 7 is a diagrammatic sectional view along the longitudinal axis of the passage channel, with an optical emitter and an acoustic wave transducer arranged inside the resonant cavity, at the upper stop, according to the invention,

FIG. 8 is a diagrammatic sectional view along the longitudinal axis of the passage channel, with an optical emitter and an acoustic wave transducer closing the resonant cavity on its base, the passage channel being arranged in the thickness of the block, according to the invention,

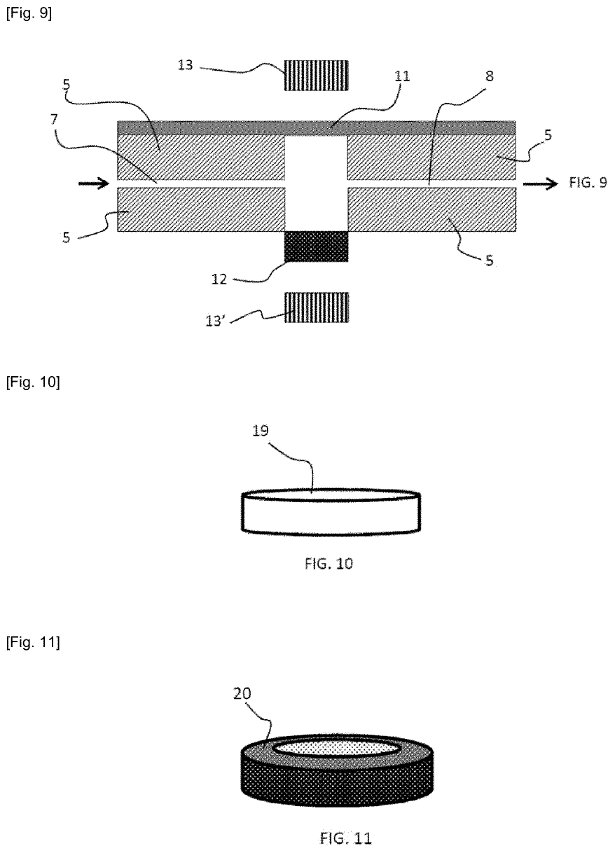

FIG. 9 is a view of the device from FIG. 8 with two optical emitters and an acoustic wave transducer, according to the invention,

FIG. 10 is a diagrammatic view of a transducer designed starting from a material which is transparent for the optical beam provided for implementing the exclusion principle, according to the invention,

FIG. 11 is a diagrammatic view of a transducer having the shape of a ring, according to the invention,

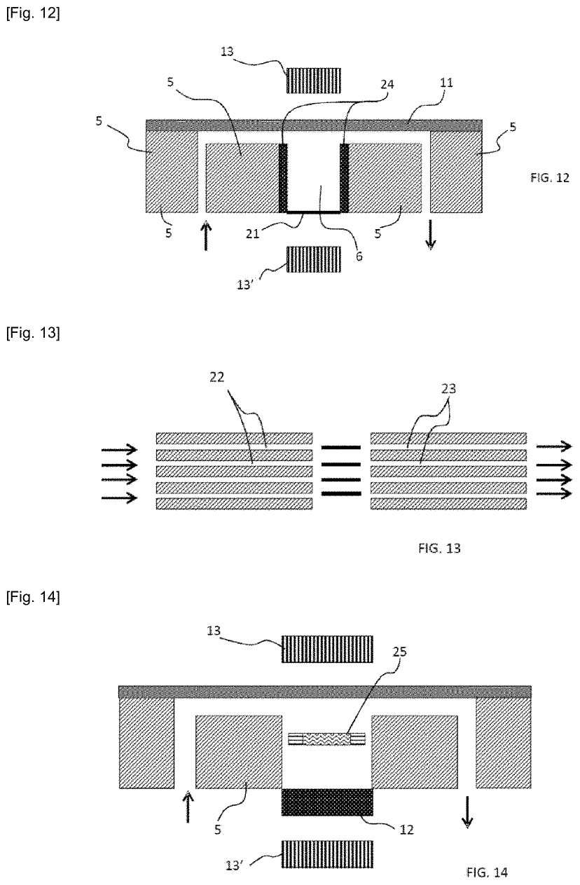

FIG. 12 is a diagrammatic sectional view along the longitudinal axis of the passage channel, with two optical emitters and a transducer with the shape of a hollow cylinder arranged around the resonant cavity, according to the invention,

FIG. 13 is a diagrammatic sectional view of a block compatible with the devices from FIGS. 2 to 12 with a plurality of microchannels etched into the block and leading into the resonant cavity, according to the invention,

FIG. 14 is a diagrammatic sectional view of a radially structured cell aggregate, according to the invention, and

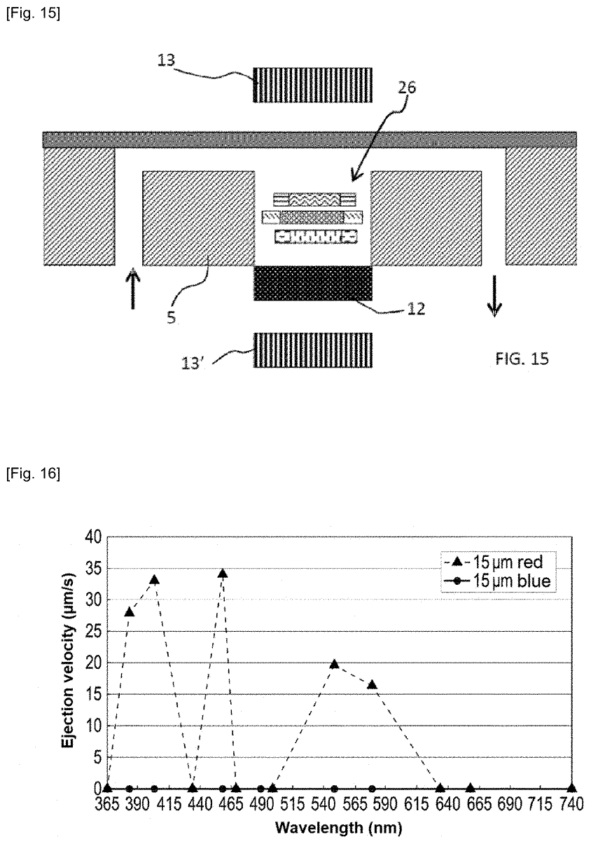

FIG. 15 is a diagrammatic sectional view of several radially and laterally structured cell aggregates, according to the invention.

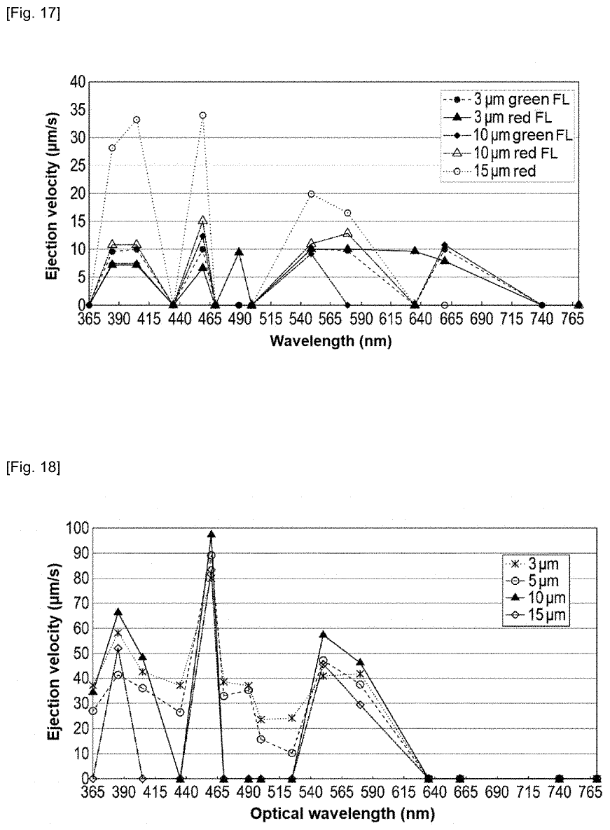

FIG. 16 is a curve illustrating ejection velocities of two types of cell as a function of the wavelength;

FIG. 17 is a curve illustrating ejection velocities of several different types of cell with different sizes as a function of the wavelength;

FIG. 18 is a curve illustrating the ejection velocity of the red blood cells as a function of the wavelength;

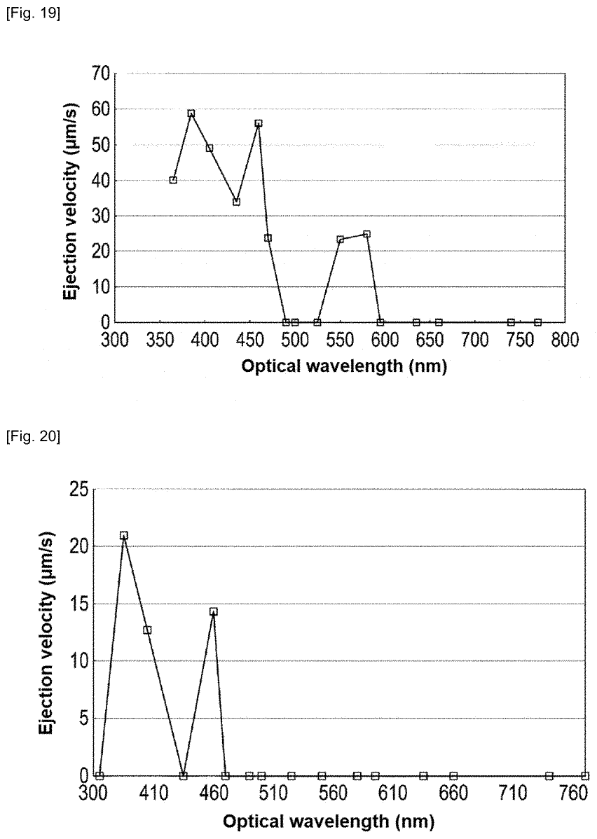

FIG. 19 is a curve illustrating the ejection velocity of the red blood cells as a function of the wavelength;

FIG. 20 is a curve illustrating the ejection velocity of the white blood cells as a function of the wavelength;

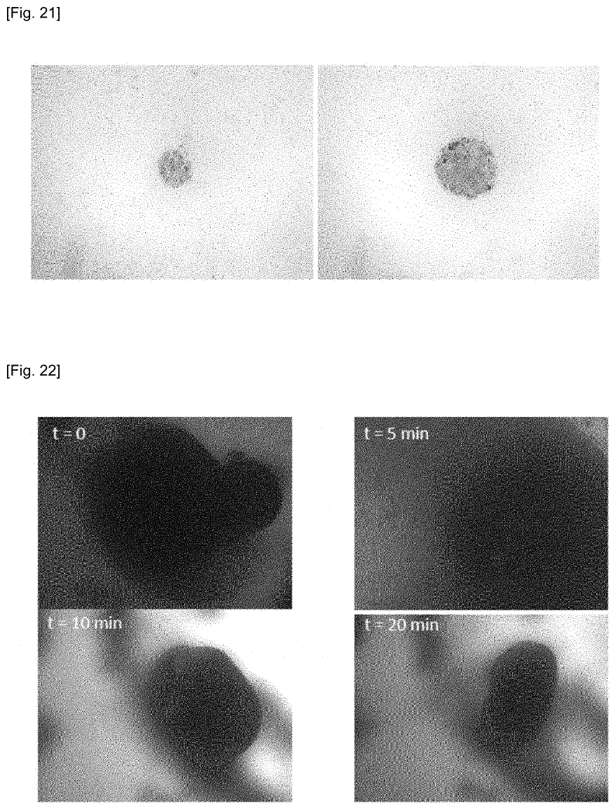

FIG. 21 comprises two photos illustrating the formation of an aggregate of white particles starting from a mixture of 10-μm particles of white polystyrene with 3-μm red particles;

FIG. 22 comprises four photos taken at different times during the formation of a “purified” aggregate in acoustic levitation by means of optical exclusion of red blood cells;

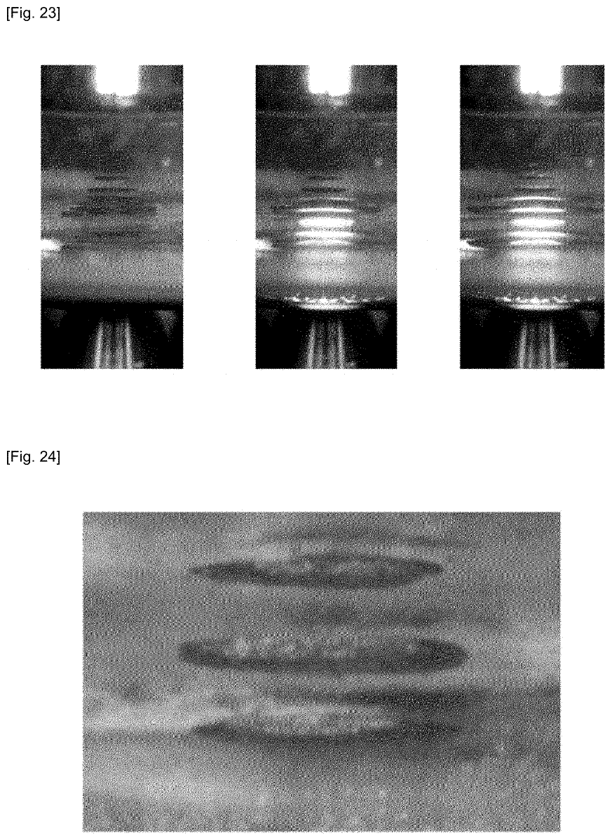

FIG. 23 comprises three photos illustrating the optical exclusion effect applied to a multi-node cavity;

FIG. 24 illustrates a layered annular structure comprising particles of red polystyrene with a diameter of 15 μm and 40-μm particles of white polystyrene.

The embodiments which will be described hereinafter are in no way limitative; variants of the invention can in particular be implemented comprising only a selection of characteristics described hereinafter in isolation from the other characteristics described, if this selection of characteristics is sufficient to confer a technical advantage or to differentiate the invention with respect to the state of the prior art. This selection comprises at least one, preferably functional, characteristic without structural details, or with only a part of the structural details if this part alone is sufficient to confer a technical advantage or to differentiate the invention with respect to the state of the prior art.

In particular, all the variants and all the embodiments described are provided to be combined together in all combinations where there is no objection to this from a technical point of view.

In the figures, elements common to several figures keep the same reference.

Although the invention is not limited thereto, a microfluidic chip will now be described which is suitable for culturing cell aggregates in acoustic levitation.

A set of components of an example of a microfluidic chip according to the invention is represented in FIG. 2. A block 5, for example made from PDMS material and with a parallelepiped shape, with an upper surface and a lower surface can be seen. A resonant cavity 6 with a cylindrical shape has been made in the centre of this block over the whole thickness of the block 5 between the two surfaces. The object of this resonant cavity 6 is to trap cells by means of the acoustic force.

For supply to the resonant cavity 6, a passage channel 7, 8 is etched on the upper surface of the block 5 such that this passage channel and the inside of the cavity are accessible. The passage channel has a first part 7 intended for the entry of the cells from an inlet microchannel 9 towards the resonant cavity. It also has a second part 8 intended for the evacuation of cells from the resonant cavity 6 towards an outlet microchannel 10. The two, inlet and outlet, microchannels are made in the thickness of the block, like the resonant cavity, and lead onto the lower surface of the block 6, the back of this block being more accessible to different devices for the supply to and management of the microfluidic chip according to the invention.

Preferably, this block is designed starting from a biocompatible material capable of ensuring gas exchanges, if necessary, between the resonant cavity and the outside (incubator). It is designed in order that the microfluidic chip according to the invention can ensure a flow of nutrients and a flow of culture medium, if necessary, within the resonant cavity. Of course, it makes it possible to inject the cells and evacuate them and makes it possible to create the aggregates with large dimensions within the resonant cavity.

The microfluidic chip is installed in an incubator so as to ensure the optimum culture conditions (gas and temperature).

A glass strip intended to cover, in particular by plasma bonding, the upper surface of the block 5 can also be seen. It can be a strip produced together with the block 5. This glass strip 11 has, at least on its internal wall facing the resonant cavity, an internal surface capable of reflecting acoustic waves from a transducer 12 provided opposite, on the side of the lower surface of the block 6. Advantageously, the glass strip is transparent to the optical beams from an optical emitter 13 arranged above the strip 11.

The strip 11 can be designed starting from one or a combination of the following materials: glass, PMMA, quartz, silicon, COC, PDMS, so as to ensure a good transmission on the one hand and a good reflection on the other.

The transducer 12 is composed of a stainless steel cylinder containing a piezoelectric element the operating frequency of which can be chosen as a function of the height of the resonant cavity. This frequency can be chosen between 0.1 MHz to 10 MHz for resonance cavities the thickness (height) of which can vary from a few mm to a few tens of μm.

FIG. 3 is a view of the block 5 from above. The ends of the microchannels 9 and 10 can be seen at each termination of the passage channel 7, 8. This passage channel has a shape that widens from the microchannels towards the resonant cavity so as to ensure a good progression of the cells when entering and when being evacuated. The passage channel can have a transverse section (circular, rectangular, square or other) with a height of from 1 to 30 mm, for example. Stops 14 and 15, as will be seen later on, are arranged on the internal wall of the resonant cavity and make it possible to define a diameter at the upper level of the resonant cavity of approximately 5 to 30 mm.

Other inlets/outlets (not represented) of the microchannel type 9, 10 and passage channel type 7, 8 can be produced for supplying the resonant cavity with identical or different cells, biomarkers, or else for washing the culture medium or recovering the production of the cells during their culturing.

An embodiment is illustrated in FIG. 4, in which the lower base of the resonant cavity is closed by an impermeable film 16. Such a configuration makes it possible to prevent any contamination of the culture medium by the transducer 12 which is located outside the cavity. This film 16 can be removable so as to make it possible to evacuate aggregates formed in the resonant cavity. Ideally, this film is transparent for the acoustic waves provided for creating the acoustic radiation force in the cavity. It can be made from PDMS, from COC or an adhesive PCR film.

The flow 17 of cells and the creation of aggregate 18 from cells trapped in the resonant cavity 6 under the action of the acoustic waves emitted by the transducer 12 are illustrated in FIG. 4.

In an embodiment such as can be seen in FIG. 5, for example, the packaged transducer 12 can be in direct contact with the culture medium in the resonant cavity. In this case, there is no transmission wall, only the glass strip 11, which is reflective, is placed opposite the transducer 12. This configuration makes it possible to improve the energy efficiency of the resonant cavity by dispensing with a transmission layer, for example the film 16 from FIG. 4, in which the energy can dissipate.

FIG. 6 illustrates an embodiment in which the transducer 12 is inserted in the resonant cavity 6. Such an embodiment makes it possible to define different volumes as a function of the application aimed at. The useful volume of the resonant cavity is then variable. The stops 14 and 15 can be arranged in different places on the internal wall of the resonant cavity so as to position the head of the transducer in predetermined positions. In FIG. 7, these stops are arranged at the top of the resonant cavity.

An embodiment is illustrated in FIG. 8, in which the passage channel 7, 8 is made completely in the thickness of the block 5 and leads into the resonant cavity at an intermediate level and not at its top. This embodiment is, of course, compatible with different configurations presented above, i.e. for example with the transducer inside or outside the cavity. However, if the transducer is inside, it must remain below the inlet of the channel 7 into the resonant cavity.

In addition to the cell culture, the manufacture of spatially structured organoids or spheroids with different layers of cells is advantageously provided.

The microfluidic chip according to the invention makes it possible in particular to inject particles or cells into the resonant cavity, and therefore to produce cell aggregates in the cavity. This makes it possible to produce cell cultures over long periods of time by providing the culture medium needed to the aggregated cells in acoustic levitation.

The invention also makes it possible to create composite and structured layers of cells, which can be useful from a tissue engineering perspective. In order to do this, the emitter 13 is used to illuminate the cells at specific wavelengths and to carry out the technique of optical exclusion.

A mixture of two types of particles or cells can be injected, these will form an aggregate which mixes the two species under the action of acoustic waves.

If it is desired to organize the aggregate spatially, in particular to structure the aggregate in successive layers, which are annular and concentric, it is possible to use the optical exclusion principle.

The optical exclusion principle makes it possible to eject particles or cells in acoustic levitation under the effect of an optical illumination at a given wavelength suitable for the cell or particle which it is desired to exclude. This effect is dependent on the optical absorption properties of the particles/cells. Cells marked with a fluorescent marker also react to an illumination at an absorption wavelength of the fluorescent marker, see in particular FIGS. 16 to 20.

The invention makes it possible to form a 2D aggregate structured in the plane by successive bands at the periphery of the aggregate. A radially structured aggregate 25 as illustrated in FIG. 14 is thus obtained. This makes it possible to manufacture a sheet composed of circular bands of cells with different natures, which can prove to be useful for the manufacture of complex organoids.

In order to do this, a mixture of two cells C1 and C2 is injected, of which one absorbs a given optical wavelength λₒₚₜ₁ and the other does not. In this case, an aggregate can be structured easily. In fact, if the aggregation area is illuminated at the wavelength λₒₚₜ₁, this will prevent C1 from aggregating under the effect of the acoustic force. The C2 species will therefore form a first aggregate. It is then sufficient to stop the illumination at the wavelength λₒₚₜ₁ in order that the C1 species forms aggregates around the first aggregate.

Using both optical and acoustic properties, therefore, it is possible to spatially structure cell aggregates in acoustic levitation. This operation can be repeated several times, by means of successive injections of different types of cells, which are marked or not, and which react to different optical wavelengths.

In FIG. 9, two optical emitters 13 and 13′ are arranged for illuminating and/or observing the cells from both sides of the resonant cavity at the same time. This thus makes it possible to exclude two species from the acoustic aggregation area simultaneously.

In order to do this, the use of a piezoelectric transducer (PZT) 19 which is transparent, as can be seen in FIG. 10, is provided. In fact, it is possible to manufacture transparent PZTs which allow the provided optical wavelengths to pass through and therefore to be free of the limitation to a single illumination from only one side.

A (non-packaged) annular transducer (PZT) can also be used. In this case, it is possible to illuminate through the ring and therefore to couple two optical sources simultaneously.

The double illumination can also be carried out with the embodiment from FIG. 12, in which a transducer 24 with the shape of a hollow cylinder is arranged around the resonant cavity. In this case, a film 21 is provided which forms the seal at the base of the resonant cavity and is transparent for the optical beams provided. In this case, the two emitters can freely illuminate the cells in the resonant cavity.

Another advantage of the culturing in levitation is that it is possible to create several pressure nodes in a cavity and thus to form cell aggregates in levitation one above another. As shown in FIG. 13, a number of nodes can be created, about fifteen for example (or several tens), in a cavity with a height of a few mm, 6 mm for example. In order to do this, several inlet microchannels 22 opposite several outlet microchannels 23 can be created. Each pair of inlet/outlet microchannels can be provided in the plane of a pressure node. However, the arrangement of cells in levitation over several pressure nodes can also be carried out with a single inlet channel, like the passage channel.

It is possible to use the optical exclusion effect on several cell aggregates in acoustic levitation and therefore to structure objects in the volume of the resonant cavity.

The aggregation area is therefore centred on the axis of the transducer. It is then possible to structure several superimposed aggregates, 26 as represented in FIG. 15, in the volume, each aggregate being able itself to be composed of several circular bands (annular structuring) of different cells. This makes it possible to envisage the manufacture, in acoustic levitation, of spheroids, organoids or tumoroids which can be held and cultured in acoustic levitation.

The creation of several monolayers one above another represents a considerable time saving for the cell culture.

The present invention therefore proposes new means for cell culturing suitable for replacing the traditional techniques of cell culturing on solid substrates. In fact, in the case of a traditional cell culturing, the cells, such as stem cells for example, will multiply on the solid substrate, but also move in order to come back into contact with the other cells. The culturing is regarded as terminated when the cells arrive “at confluency”, i.e. are in contact with one another and thus form a “monolayer” layer of cells (a single layer of cells).

The present invention relates to the design of an optoacoustic bioreactor in which the cells can be cultured in acoustic levitation and the cell aggregates can be manipulated and structured by specific illumination so as to form structured aggregates.

The inventors have shown that the opto-acousto-fluidic effect can be quantified by an ejection velocity Vₑⱼ of the illuminated objects. This involves showing that the objects in levitation according to the invention leave the illuminated area at a velocity which is in particular a function of the wavelength of the illumination signal. These objects are micro- or nanoparticles with sizes comprised between 0.1 μm and 300 μm and sensitive to the wavelengths used.

Generally, the ejection velocity can be measured for different species of particles, as a function of the optical wavelength, the intensity of the illumination, the magnification of the objective lenses of the microscope. These parameters make it possible to control the power of the illumination. In FIG. 18 for example, it is understood in particular that the size of the particle also plays an important role: the smaller the particle, the higher the ejection velocity.

FIG. 16 illustrates an ejection velocity of samples or particles as a function of the wavelength. Two groups of particles have been formed, one with red particles and the other with blue particles with a diameter of 15 μm. The curves show that the ejection velocity depends on the optical wavelength, but also on the colour of the particles. The red particles are ejected from the illuminated area for several optical wavelengths between 365 nm and 770 nm, whereas the blue particles are never ejected. In this example, the acoustic frequency is f_{ac}=0.650 MHz for an amplitude A=13 Vpp.

In FIG. 17, the following samples were analyzed under the same conditions as for FIG. 16:
3-μm green fluorescent particles,
3-μm red fluorescent particles,
10-μm green fluorescent particles,
10-μm red fluorescent particles,
15-μm red-coloured particles.

It is observed that the size of the samples influences the ejection velocity. It is also observed that the red-coloured particles have a much higher ejection velocity than the fluorescent particles.

The curve from FIG. 19 relates to the variation in the ejection velocity of the red blood cells (RBCs) (with a blood dilution of 1:1000) as a function of the wavelength. All of the experiments were carried out at a frequency of 751 kHz and an amplitude of 8 V, and the blood aggregate analyzed was placed in the centre of the cavity. It is observed that the ejection velocity of the RBCs are very high between 365 nm and approximately 490 nm, then between 515 nm and 600 nm, beyond that the red blood cells remain aggregated.

FIG. 20 relates to a curve showing the ejection velocity of the white blood cells isolated from a whole blood sample originating from a healthy patient and marked with DAPI and CD45 (λexc=415 nm, λemission=500 nm), with acoustic parameters of 751 kHz and 8 V. The maximum ejection velocity for λₒₚₜ=385 nm. The other two wavelengths to which the marked GBs react are λₒₚₜ=405 and 460 nm. The GBs do not react to other wavelengths.

The table below shows opto-acousto-fluidic responses of various cells.

| | Optoacoustic | Acoustic | | Potential |
|---|---|---|---|---|
| Cell | ejection effect | parameters | Wavelength | applications |
| Red blood cells | Yes | 746 kHz, 8 V | 365, 385, 405, | Separation of |
| | | | 435, 460, 470, | the blood cells |
| | | | 550 and 580 nm | |
| Platelets | Yes, diluted | 750 kHz, 8 V | 385, 405, 435, | Separation of |
| | (1:100) | | 460, 550 and | the blood cells |
| | | | 580 nm | |
| Neurons | No | 745 kHz, 8 V | all | Cell culture, |
| | | | | microbrain, |
| | | | | separation |
| Breast cancer | No | 745 kHz, 8 V | all | Analysis, |
| (MDA3, MCF-7 | | | | Diagnosis, |
| and SKBR3 cells) | | | | CTC recovery |
| Jurkat cells | Yes, diluted | 740 kHz, 8 V | 385, 405, 435, | Cancer |
| | | | 460, 550 and | diagnosis, |
| | | | 580 nm | CAR-T cells |
| Blood + cancer | Yes, for red | 748 kHz, 8 V | 365, 385, 405, | Separation, |
| cells | blood cells | | 435, 460, 470, | |
| | | | 550 and 580 nm | |
| WBC | yes | 745 kHz, 8 V | 385, 405, 460 | Separation, |
| | | | nm | recovery for |
| | | | | diagnosis |

FIG. 21 shows the formation of an aggregate of white particles starting from a mixture of 10-μm particles of white polystyrene with 3-μm red particles. The mixture is subjected to the acoustic force and illuminated at 460 nm (wavelength at which the red particles react), at 60% of the maximum power for a magnification of ×10.

The acoustic frequency is 1.91 MHz with an amplitude of 9 V. The flow rate is 0.15 ml/h.

The formation of an aggregate of white particles with very few trapped red particles is observed. The figure on the left corresponds to a time of 5 min, whereas the figure on the right shows the aggregate formed after 20 min. The white particles are thus gradually concentrated, with optical exclusion of the red particles.

The photos in FIG. 22 relate to different stages of forming “purified” aggregates in acoustic levitation by optical exclusion of a mixture of cells with flow. The experiment shows the evacuation of the red blood cells by applying a signal at the wavelength which makes this evacuation possible.

The cells comprise red blood cells (/100) and MDA cancer cells (/100). The illumination is obtained by a signal at 460 nm, at 80% of the maximum power for a magnification of ×10. The acoustic frequency is 1.59 MHz with an amplitude of 10 V. The flow rate is 0.15 ml/h.

FIG. 23 shows the optical exclusion effect applied to a multi-node cavity. In the photo on the left, aggregates of red particles in acoustic levitation in a microfluidic chip can be seen. The aggregates form successive layers in the shape of filled circles. In the photo in the middle, a lighting is applied which first reaches the first layer, which starts by excluding the particles from the central area so as to constitute a crown. Starting from the bottom (close to the lighting), the layers transform one after another into a crown, thus freeing up the passage of the light for the upper layer each time.

In the photo on the right, the layers are almost all transformed into crowns.

The aggregates are formed of 15-μm particles of red polystyrene. The illumination is obtained by a signal at 460 nm, at 60% of the maximum power for a magnification of ×10. The acoustic frequency is 1.91 MHz with an amplitude of 9 V. Illumination sequence: 460 off-on, i.e. first the aggregates are formed in white light (460 OFF), followed by illumination at 460 nm (ON) to form the crown of particles in acoustic levitation.

A layered annular structure can be seen in FIG. 24. The mixture of particles comprises particles of red polystyrene with a diameter of 15 μm and 40-μm particles of white polystyrene. The illumination is obtained by a signal at 460 nm, at 60% of the maximum power for a magnification of ×10. The acoustic frequency is 1.91 MHz with an amplitude of 9 V. Illumination sequence: 460 on-off, i.e. the acoustic and the optical are activated simultaneously, which excluded the red particles from the centre. The illumination at 460 nm is then switched off, which made it possible for them to form aggregates on the central aggregate.

1. A microfluidic chip capable of carrying out manipulations of cells and/or structuring and/or culturing thereof, comprising:
a block made from biocompatible material;
a passage channel made in the block for the passage of cells bathed in a liquid;
a resonant cavity made in the block, connected to the passage channel and comprising walls for containing the cells originating from the passage channel, in such a way that the cells are no longer under the influence of a flow in the passage channel;
an acoustic wave generator capable of forming at least one cell aggregate in acoustic levitation in the resonant cavity; and
at least one optical emitter capable of illuminating cells in the resonant cavity through at least one wall of the resonant cavity and simultaneous with the generation of acoustic waves in such a way as to structure said at least one aggregate by means of the technique of optical exclusion of a portion of the cells such that only the cells that are not sensitive to the illumination form an aggregate in one layer.

2. The chip according to claim 1, characterized in that it comprises at least one second optical emitter, the two emitters being arranged so as to emit through two opposite walls of the resonant cavity respectively.

3. The chip according to claim 1, characterized in that the two optical emitters emit at different wavelengths (λ_{opt 1}, λ_{opt 2}).

4. The chip according to claim 1, characterized in that the acoustic wave generator comprises an upper element and a lower element sandwiching at least part of the resonant cavity on two opposite walls; the upper element, which is fixed or removable, being an upper transducer or an acoustic wave reflector; and the lower element being a lower transducer, the transducers being capable of emitting the acoustic waves; and in that the upper element and/or the lower element being transparent to the light beams provided for illuminating the cells.

5. The chip according to claim 4, characterized in that the resonant cavity is closed at its lower end by the lower transducer.

6. The chip according to claim 4, characterized in that the lower transducer is arranged inside the resonant cavity.

7. The chip according to claim 6, characterized in that the resonant cavity has at least one stop for blocking the head of the lower transducer once it has been inserted in the resonant cavity.

8. The chip according to claim 4, characterized in that at least one of the lower transducer and the upper transducer are/is designed starting from a material which allows the optical beams provided for illuminating the cells from outside the cavity to pass through.

9. The chip according to claim 4, characterized in that at least one of the lower transducer and the upper transducer has the shape of a ring making it possible at least for the optical beams provided for illuminating the cells from outside the cavity to pass into the inside of the ring.

10. The chip according to claim 1, characterized in that the resonant cavity is closed at its lower end by a fixed or removable film which is transparent to the acoustic waves originating from the lower transducer arranged outside the resonant cavity.

11. The chip according to claim 1, characterized in that the acoustic wave generator is a transducer in the shape of a hollow cylinder arranged around the resonant cavity on the outside or forming side walls of the resonant cavity, the upper and/or lower wall of the cavity being made from a material which is transparent to the optical beams provided for illuminating the cells from outside the cavity.

12. The chip according to claim 1, characterized in that the resonant cavity is a cylinder the side walls of which are constituted by the block.

13. The chip according to claim 12, characterized in that the passage channel leads into the resonant cavity at the upper end of a side wall of the cylinder.

14. The chip according to claim 13, characterized in that the resonant cavity has a stop arranged so that the head of the lower transducer can be inserted to reduce the height of the usable volume in the resonant cavity until it is equal to the height of the passage channel.

15. The chip according to claim 1, characterized in that the passage channel is made on the upper surface of the block, a bonded or removable strip covering all of the surface of the block, including the upper end of the resonant cavity; said strip being transparent to the optical beams provided for illuminating the cells of the resonant cavity from the outside; and in that, when a transducer is arranged opposite, said strip acts as a reflector reflecting the acoustic waves from the transducer on the internal side of the resonant cavity.

16. The chip according to claim 15, characterized in that the passage channel and the cylinder are arranged perpendicular to one another, and in that the block moreover comprises two microchannels passing through the block from one side to the other, parallel to the cylinder and connected respectively to the two free ends of the passage channel; the first microchannel being intended for the arrival of cells in the passage channel and the second microchannel being intended for the evacuation of cells from the passage channel.

17. The chip according to claim 4, characterized in that the reflector is a strip made from glass, from polymethyl methacrylate (PMMA), from quartz, from silicon, from polydimethylsiloxane (PDMS) or from cyclic olefin copolymer (COC).

18. The chip according to claim 4, characterized in that the reflector is designed starting from a material identical to that of the block and has an internal surface treated to reflect acoustic waves.

19. The chip according to claim 1, characterized in that it comprises several microchannels made in the thickness of the block and leading into the resonant cavity for cells to enter and/or exit.

20. The chip according to claim 1, characterized in that the height of the resonant cavity is a function of the number of pressure nodes to be created and the wavelength of the acoustic waves generated by the generator.

21. The chip according to claim 1, characterized in that the block is made from polydimethylsiloxane (PDMS) or from cyclic olefin copolymer (COC).

22. The chip according to claim 1, characterized in that the resonant cavity is dimensioned with a height greater than the diameter of the passage channel leading into this resonant cavity.

23. The chip according to claim 1, characterized in that the resonant cavity has a diameter between 1 and 50 mm, the height of the resonant cavity being comprised between 5 and 15 mm and the height of the passage channel being equal to 450 μm.

24. The chip according to claim 1, characterized in that it moreover comprises at least one additional microchannel made in the block in the same plane as the passage channel.

25. A method for manipulating cells in acoustic levitation in a microfluidic chip according to claim 1, this method comprising the following steps:
injecting cells into the resonant cavity via an inlet of the passage channel;
generating acoustic waves for acoustically levitating the injected cells so as to form a cell aggregate in at least one layer; and
at least one phase of illuminating the cells while simultaneously maintaining the acoustic waves so as to manipulate cells according to the optical exclusion principle.

26. The method according to claim 25, in which method the injected cells have different natures, the steps of generating acoustic waves and of illuminating being carried out as follows:
generating acoustic waves for acoustically levitating the injected cells and at the same time applying a light beam at a wavelength making the principle of exclusion of a portion of the cells possible, so that only the cells not sensitive to this optical wavelength form an aggregate in one layer; and
maintaining the acoustic waves and stopping the light beam so that the cells sensitive to the wavelength of the light beam now form aggregates on the periphery of the aggregate already formed, to thus obtain a radially structured aggregate.

27. The method according to claim 25 for producing a three-dimensional structure formed of several layers of aggregates:
the step of injecting comprising the injection of cells into the resonant cavity via one or more inlets; and
the step of generating acoustic waves moreover comprising the generation of acoustic waves for acoustically levitating several aggregates of cells injected on several levels, the levels being acoustic pressure nodes the number of which is a function of the wavelength of the acoustic waves and the height of the resonant cavity.

28. The method according to claim 25, characterized in that it moreover comprises a step of carrying out a cell culturing while holding the aggregate or the aggregates obtained immobile in acoustic levitation for the duration of the culturing.
